(19) United States
(12) Patent Application Publication
(10) Pub. No.: US 2005/0186287 A1
(43) Pub. Date: Aug. 25, 2005

(54) Copper agonist that binds on the copper binding site of APP and/or exerts an inhibiting effect on the release of amyloid Abeta peptide

(76) Inventors: Konrad Beyreuther, Heidelberg (DE); Gerd Multhaup, Dilsberg (DE); Colin L. Masters, Victoria (AU)

Correspondence Address: BURNS DOANE SWECKER & MATHIS L L P, POST OFFICE BOX 1404, ALEXANDRIA, VA, 22313-1404, US

(21) Appl. No.: 10/848,016

(22) Filed: May 19, 2004

Related U.S. Application Data
(62) Division of application No. 09/914,770, filed on Jan. 2, 2002, now abandoned.

(30) Foreign Application Priority Data
Mar. 3, 1999 (DE) 199 09 357.1

(51) Int. Cl. G01N 33/53; G01N 33/567; A61K 33/24; A61K 38/40; A61K 48/00

(52) U.S. Cl. 424/617; 514/2; 514/44; 435/7.2

(57) ABSTRACT

The invention relates to copper agonists that bind on the copper binding site of APP and/or exert an inhibiting effect on the release of the amyloid Aβ peptide participating in the emergence of Alzheimer's disease. The invention also relates to the drugs containing said copper agonists or to the utilization of said copper agonists for the preparation of a drug for the prevention and/or treatment of Alzheimer's disease. The invention further relates to a method for identifying copper agonists having the desired effect described in the invention.

The present invention relates to copper agonists which bind to the copper binding site of the amyloid precursor protein (APP) and/or exert an inhibitory effect on the release of the amyloid Aβ peptide which is involved in the development of Alzheimer's disease. The present invention also relates to medicaments containing these copper agonists for preventing and/or treating Alzheimer's disease. Finally, this invention concerns methods of identifying copper agonists having the effect desired for the purposes of the invention.

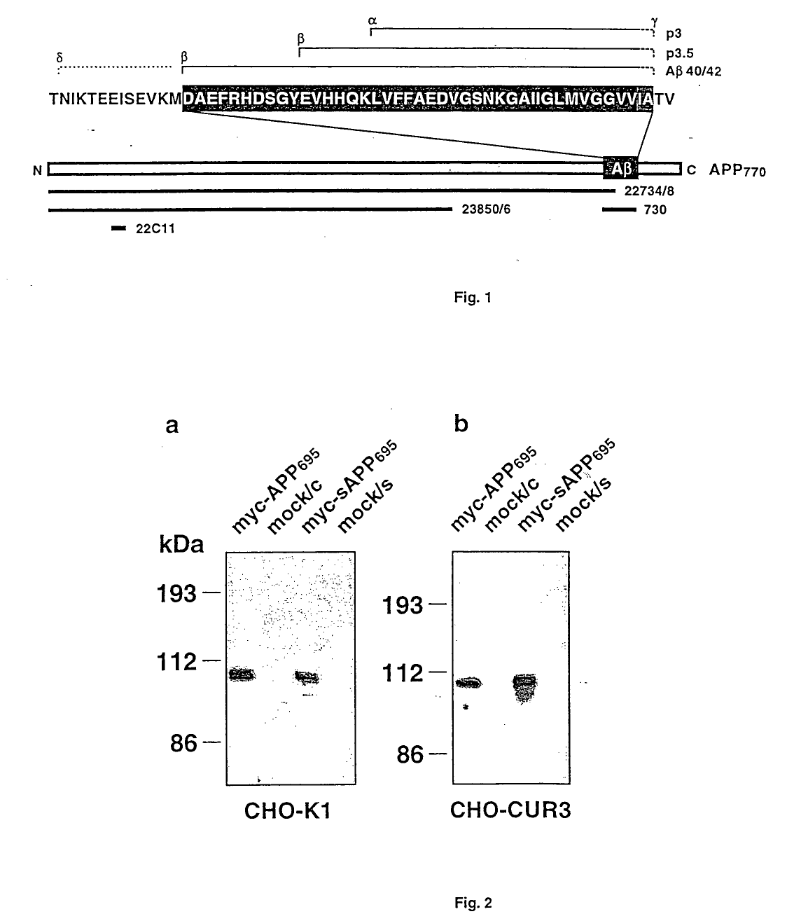

The β amyloid peptide (βA4, Aβ), the basic component of senile plaque and the cerebrovascular amyloid of Alzheimer's disease form by successive proteolytic cleavages with β-secretase and γ-secretase from the larger integral membrane amyloid precursor protein (APP) whose main isoforms contain 695 (APP695), 751 (APP751) or 770 (APP770) amino acids (Hardy, J., Trends Neurosci 20, pp. 154-159 (1997)). The APP processing within the Aβ domain by a-secretase prevents amyloid formation and leads to the release of the p3 peptide which contains the Aβ residues 17-40/42 (FIG. 1). The cleavage of APP with α-secretase or β-secretase produces soluble APP fragments (sAPPα and sAPPβ) which represent the APP ectodomains supplied to the extracellular space.

Although the physiological role of APP is not known, APP shares some characteristics with cell adhesion molecules and with molecules involved in wound healing. This comprises the binding sites of APP for heparin sulfate, collagen, laminin, proteases, lectins and metal ions, for example.

In the former experiments of treating-Alzheimer's disease, the formation of the amyloid Aβ peptide was blocked by administering compounds blocking the hitherto unidentified β-secretase or γ-secretase (Hooper et al., Biochem. J. 321, pp. 265-279 (1997)). However, the inhibition of these proteases leads to undesired side effects, since these proteases are involved in the processing of different transmembrane proteins. The treatment of Alzheimer's disease and other forms of dementia has focused thus far on the improvement of the metabolic processes in the brain and the substitution of certain deficitary neurotransmitters or on the inhibition of the inflammatory processes accompanying this disease. However, these medicaments do not make a distinction between the different forms of dementia but aim at the secondary symptoms which can be observed in all dementias.

It is thus the technical problem of the present invention to provide products which are of use for preventing or treating Alzheimer's disease.

This technical problem has been solved by providing the embodiments characterized in the claims.

It has been found that APP interacts specifically with Zn(II) and Cu(II) at two defined locations. The binding of Cu(II) leads to an oxidation of two cysteines of APP, which results in the formation of an additional cystine. One of the two electrons produced in this way reduces the bound copper(II) to form copper(I). However, the fate of the other electron has not been elucidated thus far.

It has now turned out that the addition of copper(II) effects in mammalian cells an increase in the release of the APP products of the α-secretory metabolic pathway (sAPPα and p3) and a decrease in the release of the products of the β-secretory metabolic pathway (p3.5 and the Aβ involved in Alzheimer's disease). Alzheimer's disease can thus be prevented or treated effectively and specifically by administering compounds which act as copper agonists, i.e. which can bind to the copper binding site of APP and/or imitate its physiological effect, i.e. can reduce or prevent the formation of the amyloid Aβ peptide. Since the copper binding by APP also leads to the production of very toxic Cu(I) ions, these molecules with an agonistic effect comprise the additional characteristic of preventing the cells from damage by the Cu(II) binding of APP and are thus able to prevent the formation of reactive forms of oxygen produced by copper and hydrogen peroxide.

Thus, an embodiment of the present invention relates to a copper agonist which is characterized in that it binds to the copper binding site of the amyloid precursor protein (APP) and/or reduces or prevents the release of the amyloid Aβ peptide.

The term “copper agonist” used herein concerns any substance, e.g. an inorganic or organic compound, which can bind to the copper binding site of APP and/or can reduce or prevent the release of the amyloid Aβ peptide. Preferred copper agonists are the divalent metal ions which may compete with copper for the binding site at the APP molecule at physiologically high concentrations, e.g. 20-200 mM Mg(II), preferably 100 mM Mg(II), 20-200 mM Ca(II), preferably 100 mM Ca(II); 0.05-20 mM Zn(II), preferably 100 μM Zn(II), etc. Further preferred copper agonists are oligopeptides, oligonucleotides, oligosaccharides or nucleotide analogs which imitate copper mainly via their three-dimensional structure and bind to the copper binding site according to the key-and-lock principle. In a preferred embodiment, the oligopeptides may also contain a sequence. corresponding to the copper binding site and thus occupy it. Further preferred copper agonists are obtained from purchasable chemical substance libraries or are new chemical compounds (combined chemistry) or low molecular natural substances isolated from microorganisms or plants. These compounds also act via their three-dimensional structure which imitate the characteristics and size of copper without having its negative properties.

The effect as copper agonist is thus based preferably on the fact that the substance binds specifically and with an affinity higher than that of copper ions to the Cu binding site of APP or binds specifically to the sequence region of APP, which is responsible for copper binding and prevents (sterically) a further copper binding at that place or drives the copper ions from that place. Copper agonists are competitive (for the copper binding site) and non-competitive binding ligands which can imitate the physiological effect of copper. Copper agonists within the meaning of the present invention are also those substances which do not bind to the copper binding site but stabilize e.g. the conformation of APP which is characteristic of the copper APP complex, i.e. they can imitate the physiological effect of copper at other binding sites (beyond the direct copper binding site).

The term “reduces or prevents the release of the amyloid Aβ peptide” used herein relates to an effect of the copper agonist which can be referred to as sufficient to obtain a preventive or therapeutic effect as regards Alzheimer's disease.

A person skilled in the art is capable of identifying and producing substances having the above described effect as copper agonists by means of common methods used in the art. In the case of non-natural agonists (e.g. small organo-chemical synthesized substances available as what is called libraries), oligonucleotides, oligopeptides and nucleotide analogs, the synthesis is made according to common methods sufficiently known to the person skilled in the art. The identification of the substances as possible agonists can be made via affinity chromatography, e.g. using the “BIAcore technique”. In this case, the above-mentioned compounds are tested for binding to APP, in particular to the domain of APP which comprises the copper binding site. Thereafter, the identified ligands are tested for an effect on the APP processing in the cell culture system, e.g. by means of competition assays with suitable antibodies. Moreover, the person skilled in the art can also test by means of common methods, e.g. in analogy to the methods described in Examples 4 and 5, whether the identified agonists can be tolerated by the patient and optionally prepare a dose-effect curve. The method according to the invention for identifying a copper agonist binding to the copper binding site of APP and/or exerting an inhibitory effect on the release of the amyloid Aβ peptide is therefore characterized by the following steps:
(a) contacting APP or a fragment thereof carrying the copper binding site with various concentrations of a compound which potentially has the above effect,
(b) detecting a decrease of the Aβ protein.

In a preferred embodiment, the method is characterized by the following steps:
(a) contacting of APP or a fragment thereof carrying the copper binding site with a dissolved or immobilized substance library-or with low molecular substances from microorganisms and/or plants,
(b) when a dissolved substance library or liquid low molecular substances are used, immunoprecipitation of the copper binding site/ligand complex from the solution with antibodies specific to APP or the fragment thereof or, when an immobilized substance library is used, release of the ligand from the copper binding site/ligand complex by the addition of copper salts,
(c) identification of the ligand, and
(d) selection of ligands which by the competitive or non-competitive binding to the copper binding site of APP or to other sites of APP exert an inhibitory effect on the release of the amyloid Aβ peptide,
wherein step (d) can optionally precede step (c).

A “ligand” according to the invention is understood to mean every substance which binds to the copper binding site or other sites of APP in the course of the above process.

The above mentioned antibodies are e.g. monoclonal or polyclonal antisera recognizing APP and suitable for immunoprecipitation. They can be purchased, e.g. from the companies of Boehringer Mannheim, Dianova or Sigma. Further suitable antibodies which can be used for detecting APP, APP complexes and Aβ40 (isoform of Aβ having 40 amino acids, see FIG. 1) or Aβ42 (isoform of Aβ having 42 amino acids, see FIG. 1) are described in Ida et al., J. Biol. Chem. 271, pp. 22908-22914 (1996) and Weidemann et al., Cell 57, pp. 155-162 (1989).

Collections of organo-chemical compounds which formed around lead substances due to random synthesis are used as substance libraries. These libraries can be purchased (e.g. from Morphosys, Munich; Analytikon, Berlin) or can be produced cooperatively by chemical laboratories (e.g. new chemical compounds by combined chemistry). Low molecular substances from microorganisms and plants can be isolated using the BIAcore technique.

Contacting is effected either in solution or with immobilized APP or with fragments of. APP. The complexes formed are precipitated from the solution using antibodies and then detected using fluorescence, radioactivity or analytical methods, such as mass spectroscopy, gas chromatography, etc.

A suitable APP fragment is determined by initially testing the binding of the test substances to the entire APP and subsequently testing more and more shorter fragments which have the same binding characteristics as the full-length molecule.

Another possibility of carrying out step b) is by means of affinity chromatography, e.g. BIAcore, i.e. either APP molecules or its ligands are immobilized on a carrier surface. Thereafter, the corresponding molecule is injected onto this surface and when a binding has taken place copper salts are injected to resolve the binding again. This process can be pursued directly by the biosensor technique.

In a preferred embodiment of the identification method according to the invention, step (d) comprises the incubation of mammalian cells transfected stably with human APP695 with the ligands obtained from steps (a) to (c) and the determination of the production of the amyloid Aβ peptide with polyclonal or monoclonal antibodies. In this case, immunoprecipitation with subsequent SDS gel electrophoresis, ELISA and immunoprecipitation Western blots are in consideration as assays.

As an alternative, it is possible to proceed such that in step (d) the ligand obtained from steps (a) to (c) is administered to transgenic mice expressing the human amyloid Aβ peptide. Thereafter, a sample is collected from the CNS or the blood of the caudal vein and the production of the amyloid Aβ peptide with polyclonal or monoclonal antibodies is determined (e.g. companies of Dianova, Boehringer Mannheim, Sigma and own antibodies [Ida et al., J. Biol. Chem., 271, pp. 22908-22914, 1996]). This alternative method represents an in vivo test of the identified ligands. The procedure (including the methods used) is here very similar to the above described cell culture experiment. In place of detecting the APP fragments formed in the cell culture supernatant or intracellularly, in mice these fragments are detected in the serum. The detection methods correspond to those of the cell culture experiment.

The present invention also relates to medicaments containing the copper agonists according to the invention. These medicaments optionally contain in addition a pharmaceutically acceptable carrier. Suitable carriers and the formulation of such medicaments are known to the person skilled in the art. Suitable carriers are e.g. phosphate-buffered common salt solutions, water, emulsions, e.g. oil/water emulsions, wetting agents, sterile solutions, etc. The medicaments can be administered orally or parenterally. The methods for the parenteral administration comprise the topical, intra-arterial, intramuscular, subcutaneous, intramedullary, intrathekal, intraventricular, intravenous, intraperitoneal or intranasal administration. A suitable dosage is determined by the attending physician and depends on various factors, e.g. on the age, sex and/or weight of the patient, the stage of the disease, the kind of administration, etc.

Thus, the present invention also relates to the use of the above described copper agonists for the production of a medicament for preventing or treating Alzheimer's disease.

The invention is described in more detail by means of the figures according to which:

FIG. 1 shows the processing of the amyloid precursor protein (APP770) into Aβ, p3.5 and p3.

One-letter code; the white letters designate the amino acid sequence of Aβ1-40/42. The approximate position of the epitopes for the antibodies is indicated below the sequence with black bar.

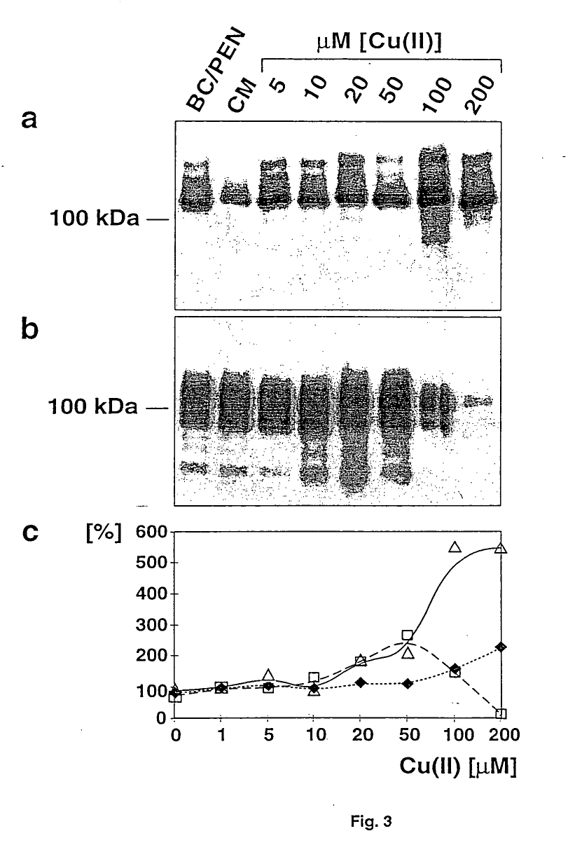

FIG. 2 shows the immunoprecipitation of c-myc-tagged APP695

Immunoprecipitation was made with polyclonal c-myc antibodies 18/47 from the lysate (myc-APP695) and the conditioned medium (myc-sAPP695) of the wild type (a, CHO-K1) and from copper-resistant CHO cells (b, CHO-CUR3). The precipitated samples were immunoblotted with the monoclonal APP antibody 22C11 after being transferred to a nitrocellulose membrane. Control: medium (“mock/s”) and lysate (“mock/c”) or “mock”-transfected cells.

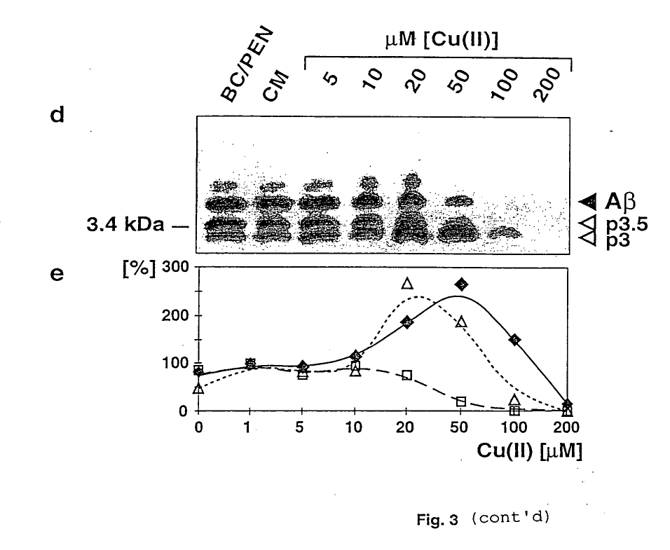

FIG. 3 shows the immunoprecipitation of Holo-APP695 from lysate (a), soluble APP695 (b) and Aβ and p3 from medium (d) of metabolically labeled CUR3 cells with anti-APP (22734/6; polyclonal serum from rabbits which was obtained by immunization with APP produced in bacteria, see FIG. 1) or anti-Aβ (730; polyclonal serum from rabbits which was obtained by immunization with synthetically produced Aβ40 peptide) after 4-hour incubation in the presence of the copper chelating agents bathocuproin (BC) and D penicillamine (PEN) in common medium (CM; containing basal medium-copper) or in medium which contained the indicated copper concentrations

The appearance of sAPP695 as a doublet (b) at higher copper concentrations is based on its different sialic acid content. A fragment (d) running somewhat above the Aβ band occurs with low copper concentrations and is presumably produced by cleavage with 6-secretase and γ-secretase (see also FIG. 1). The relative amounts of cAPP (open triangles), sAPP (open squares) and entire secreted protein (black squares), which were measured in this experiment, were quantified as percentage of radioactivity, obtained with basal medium copper concentrate in c); Aβ (open squares), p3 (open triangles) and sAPP (black squares; the data originating from the same experiment as in (c) are shown for better comparison) in (e). The data correspond to the average values from three independent experiments. “0” μM and “1” μM copper in (e) refer to incubation conditions for removal of copper (PEN/BC) and copper in the basal medium (CM; 0.8 μM).

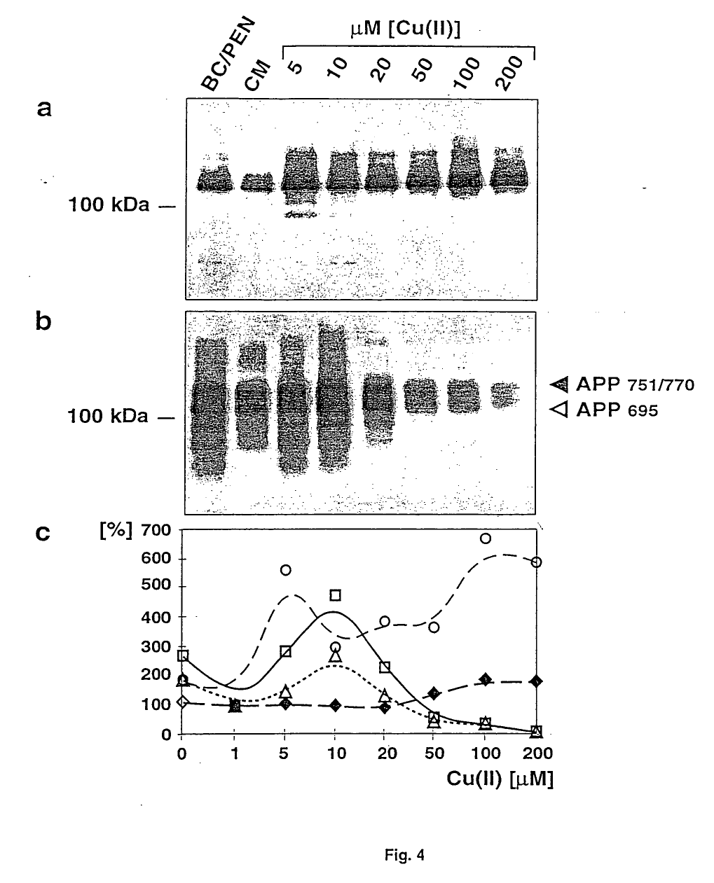

FIG. 4 shows the immunoprecipitation of Holo-APP695 from lysate (a), soluble APP695 (b) and Aβ and p3 from medium (d) of metabolically labeled K1 cells with anti-APP (22734/6) or anti-Aβ (730) after 4-hour incubation in the presence of the copper chelating agents bathocuproin (BC) and D penicillamine (PEN) in common medium (CM; containing basal medium-copper) or in medium which contained the indicated copper concentrations; quantified as in FIG. 3.

The endogenous level of sAPP751/770 is higher in K1 cells than in CUR3 cells (see FIG. 2) and is also influenced by the copper concentration and was thus quantified. The data are average values from two independent experiments. The relative amounts of cAPP (open circles), sAPP695 (open squares), sAPP751/770 (open triangles) and an entire secreted protein (black squares), which are measured in this experiment, are quantified in c). Aβ (open squares), p3 (open triangles) and sAPP695 (black squares; the data of the same experiment shown in c) in (e). “0” μM and “1” μM copper in (e) refer to incubation conditions for the removal of copper (PEN/BC) and copper in basal medium (CM; 0.8 μM). A fragment (d) somewhat running above the Aβ band occurs with low copper concentrations and is presumably produced by cleavage with δ-secretase and γ-secretase (see also FIG. 1).

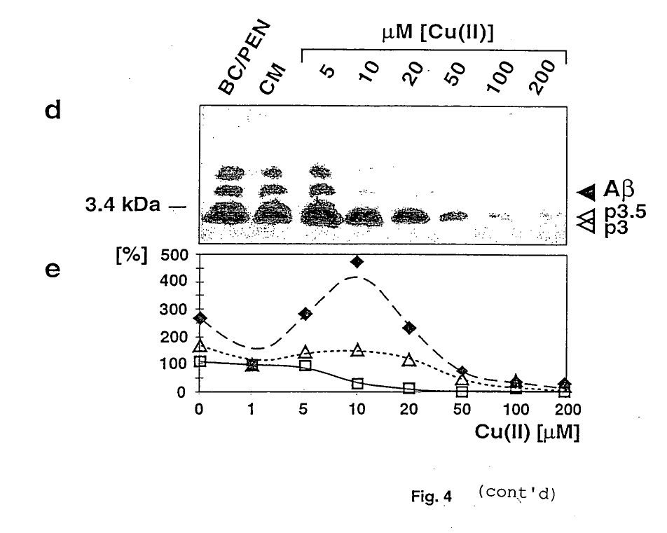

FIG. 5 shows the quantification of the relative levels of sAPP695, p3 and Aβ which were immunoprecipitated from conditioned medium of CHO-K1 cells (a) and CHO-CUR3 cells (b)

The increase in sAPP695 (black squares) achieved a maximum at 10 μM Cu(II) (a) in K1 cells and at 50 μM Cu(II) (b) in CUR3 cells; this was followed by a decrease. The change in the sAPP level (open triangles) occurred later as compared to the changes with the p3 protein which reached the maximum level with somewhat lower Cu(II) dosages, which is most likely due to a half life longer as compared to sAPP. In contrast thereto, the Aβ levels (black squares) increased drastically in both cell lines with Cu(II) concentrations below the basal level. The first data point was determined with the basal copper concentration (0.8 μM Cu(II)).

The below examples illustrate the invention.

### EXAMPLE 1

### General Methods

Cells Lines and Transfections

CHO cells were transfected with a c-myc-tagged APP695 vector (Peraus et al., J. Neurosci 17, page 7714-7724 (1997)) or with the expression vector (pcDNA3) by means of a highly efficient calcium phosphate transfection (Chen et al., Biotechniques 6, pages 632-638 (1988). The pSP65 (Invitrogen company) N-tag-APP 695 DNA was cloned into the vector pcDNA3 (Invitrogen/ITC Biotechnology, Heidelberg) using the SmaI or EcoRV cloning sites. The transfection efficiency was checked by means of immunoprecipitation of APP with polyclonal rabbit-anti-c-myc antibody (18/47; produced against the amino acid sequence EQKLISEEDL of the c-myc sequence; from Eurogentec, Seraing, Belgium) and immunoblots using the monoclonal mouse antibody 22C11 (Boehringer Mannheim) in the “ECL” detection system (Amersham, Braunschweig). The parental CHO-K1 cells and the copper-resistant cell variant CUR3 were cultured at 37° C. in “Eagles” medium (BME), to which 2 mM L-glutamine, 0.1 mM proline, 20 mM HEPES and 10% fetal calf serum (Boehringer Ingelheim) were added. The basic copper concentration was 0.8 μM. CUR3 cells were cultured in a medium with 200 μM of copper added.

Metabolic Labeling and Immunoprecipitation

Stably transfected CHO cells of a culture dish (60 mm) were treated for 4 hours with 2 ml essential minimum medium (MEM) which contained no methionine (Sigma company, Munich) and was supplemented with 220 μCi [³⁵S]methionine (Amersham company, Braunschweig) and 5% N₂. The conditioned medium (CM) and the cells were collected and immunorepcipitated. The protein concentrations were determined beforehand by means of a “BioRad” protein assay (Bradford et al., Anal. Biochem. 72, pages 248-254 (1976)) or the radioactively labeled proteins were precipitated with 10% trichloroacetic acid (TCA) and the incorporated [³⁵S]methionine was measured (Beckman LS 6000IC). The cells were lyzed in extraction buffer which contained 50 mM Tris-CHl (pH value 7.5), 150 mM NaCl, 2 mM EDTA, 2% triton X-100, 2% NP40, 10 μg/ml aprotinin and 10 μg/ml leupeptin. For removing cell debris, the lysate and the medium were centrifuged for 10 minutes at 13,000×g and the medium was adjusted to 25 mM Tris-CHl (pH value 8.5), 1 mM PMSF, 10 μg/ml aprotinin, 10 μg/ml leupeptin, 0.5% triton X-100 and 0.5% NP40. The solubilized proteins were diluted in 100 mM Tris-HCl (pH value 7.5), 300 mM NaCl and 4 mM EDTA at a concentration of 1:2. The supernatants were incubated overnight at 4° C. with APP antibodies with end-over-end rotation in an overhead shaker. In some experiments, the resulting supernatants were then analyzed using Aβ antiserum. Immunocomplexes were obtained with protein A sepharose and analzyed as described in (Weidemann et al., Cell 57, pages 115-126 (1989)).

Antibodies and Electrophoresis

For detecting APP, polyclonal rabbit antibodies directed against recombinant Fd-APP770 (antiserum 22734/6 against the amino acid residues 18-687 of APP770 or antiserum 23850/6 against the APP amino acid residues 18-491 (FIG. 1) were diluted at a concentration of 1:500 for precipitation. The antisera were produced in a laboratory according to standard methods known to the person skilled in the art. Aβ, p3.5 and β3 were recognized by the polyclonal antibodies 830 (diluation 1:50; Aβ, p3.5 and p3 were likewise precipitated) which had been produced against a synthetic peptide corresponding to the Aβ amino acid residues 1-40 (FIG. 1). Aβ and its derivatives were separated by 10-20% Tris-tricin-polyacrylamide gels and APP using 7% Tris-glycin-polyacrylamide gels or 7% Tris-tricin-polyacrylamide gels. For immunoblots cell extracts and extracellular medium were precipitated with 10% TCA, washed with acetone and dissolved in sample buffer. After the electrophoresis, the gels were further processed as described in (Simons et al., J. Neurosci 16, pages 899-908 (1996)) and quantified with the “Fuji-Bas-PhosphorImager” system. The data were expressed as average values +/− standard deviation and, unless indicated otherwise, they represent the result of at least two separate experiments.

Treatments of Active Substances and LDH Assay

During the labeling period copper chloride or zinc chloride (concentrations: 5 μM to 200 μM) were added to the culturing medium. D(−)-penicillamine (PEN; Sigma), bathocuproin-disulfonate (BC; Aldrich) and 1,10-phenanthroline (PEN; Sigma) were added to the medium up to a final concentration of 100 μM (PEN and BC) or 200 μM (PEN). The viability of the cells was measured by means of the lactate dehydrogenase (LDH) outflow into the culture (41). The LDH outflow was measured as enzyme activity (units/liter) after 2 and 4 hours in the medium of 70% confluent monolayers of CHO cells in “Eagle” medium modified according to Dulbecco (DMEM)

### EXAMPLE 2

### APP Expression in Cells Which Were Transfected With APP695-Encoding cDNA

The parental CHO-K1 cell line and the copper-resistant CUR3 cell line were used for studying the role of copper on APP processing with decreasing copper concentrations or increasing copper concentrations (0-200 μM). CUR3 cells were used for investigating the role of intracellular copper on APP processing. As compared to CHO-K1 cells, CUR3 cells show lower intracellular copper concentrations, which is caused by an enhanced outflow of copper as a result of a concentration, increased by a factor of 70, of the “Menkes P-type ATPase copper efflux pump” (intracellular copper pump/intracellular copper export system).

Following the transfection of CHO-K1 and CUR3 cells with cDNA, which encoded APP695 with an N-terminal c-myc epitope (FIG. 1), APP expression could be detected easily by means of immunoprecipitation with polyclonal c-myc antiserum and subsequent immunoblots with the monoclonal antibody 22C11 (FIG. 2a,b). The polyclonal c-myc antibody immunoprecipitated c-myc-tagged APP695 with a relative molecular weight of 105 kDa from the cell lysate (cAPP695) and the conditioned medium (sAPP695). In accordance with expectations, c-myc-APP695 could not be detected in either the lysate or the medium of cells which had only been transfected with pcDNA3.

### EXAMPLE 3

### Decrease of the Aβ Concentration as a Function of Increasing Copper Concentrations

For investigating the regulation of the APP metabolism by copper ions, CHO cells stably transfected with APP695 were labeled for 4 hours with ³⁵S-labeled methionine in the presence of different copper concentrations and following immunoprecipitation with polyclonal antibodies which detect APP (22734/6) or detect Aβ and p3 (730), the secretions of the APP ectodomain, of Aβ and p3 were measured. Cell lysates of CUR3 cells showed a band at 105 kDa which corresponds to the immature cAPP695 holoprotein (FIG. 3a). APP695 derivatives released in conditioned medium were detected as bands of 105 and 97.5 kDa (FIG. 3b), they probably differing in that the upper band has a higher content of sialic acids. The cell lines showed no reduced viability during the entire experiment.

When CUR3 cells with increasing copper concentrations were incubated, the concentration of the APP holoprotein and of soluble APP also increased significantly. As compared to the basal medium, a maximum of sAPP was obtained with 265% at 50 μM Cu(II) before a general production of secreted proteins was induced (FIG. 3c; Table 1). The secretion of p3, the C-terminal counterpart of secretory APP which is produced by cleavage with α-secretase, increased between 20 μM and 50 μM in the medium to 270% (FIG. 3d; Table 1). The immunoprecipitation of the radioactively labeled peptide originating from the Aβ region of APP was made with the rabbit antiserum 730, which recognizes Aβ (4.5 kDa), p3.5 (3.5 kDa) and p3 (3 kDa) (FIG. 1). When a copper concentration of 50 μM was exceeded in the medium, this influenced the general protein metabolism, and the percentage of cAPP was markedly higher with 550% (FIG. 3c; Table 1). In contrast thereto, the Aβ concentration was strongly lowered, only 20% of the basal concentration were present with 50 μM copper and above 100 μM copper Aβ was hardly detectable in CUR3 cells (FIG. 3d,e). This was also determined for p3.5 (FIG. 3d), which confirmed the earlier observation that p3.5 is the product of an alternative β-secretase metabolism.

TABLE 1
Modulation of APP processing in CHO cells*

| | SAPP695c | APP695 | p3 | Aβ |
|---|---|---|---|---|
| CUR3 (50 μM Cu(II)) | 265 | 550 | 270 | 20 |
| CUR3 (Cu(I)/Cu(II) removal) | 77 | 205 | 50 | 100 |
| K1 (10 μM Cu(II)) | 475 | 295 | 154 | 20 |
| K2 (Cu(I)/Cu(II) removal) | 170 | 190 | 171 | 100 |

*[%] increase/decrease as compared to standard conditions

### EXAMPLE 4

### Stimulation of the Aβ Peptide Production by Complex Formation of Copper(I) Ions with Bathocuproin or the Copper(II) Ions by Penicillamine

The following investigations were made to check whether cAPP, sAPP and subsequent degradation products are modulated by a copper deficiency. In order to study whether copper(II) is necessary for APP expression and processing, copper(II) was removed by means of the copper(II) chelating agent D-penicillamine (PEN) and copper(I) was removed by means of the cell-impermeable copper(I) chelating agent bathocuproindisulfonic acid. It showed that the production of Aβ and p3.5 was not influenced whereas the concentrations of sAPP and p3 were lowered to 77% and 50%, respectively (FIG. 3d,e; Table 1). This shows that the copper(II)-induced changes are based on a specific modulation of the APP metabolism by copper.

### EXAMPLE 5

### Study of the Endogenously Expressed KPI-Containing APP751/770 Isoforms in CHO-K1 Cells

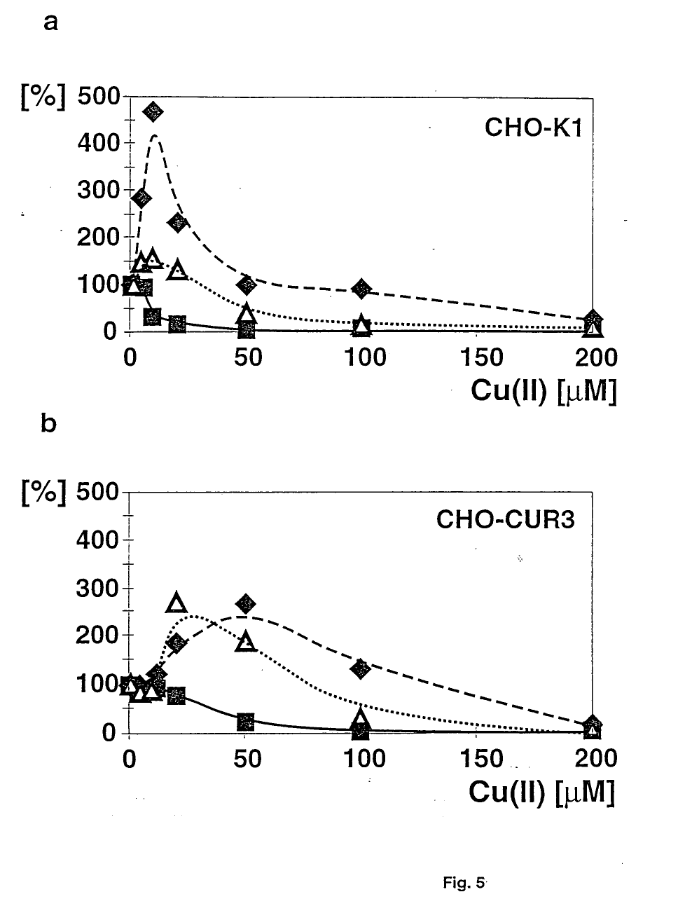

The endogenously expressed isoforms APP751/770 containing KPI (protease inhibitory domain of the Kunitz type, which is introduced into APP751 and APP770 by alternative splicing) were studied in CHO-K1 cells by immunoprecipitation of proteins labeled with [³⁵S]methionine from the cell lysate and conditioned medium with polyclonal APP antiserum 22734 (FIG. 1; FIG. 4a,b). The secretion of KPI-AAP corresponded to that of c-myc APP695 (FIG. 4). Two main bands with a relative molecular weight of 130 kDa and 105 kDa correspond to sAPP751/770 and sAPP695, respectively. The latter could also be precipitated with anti-c-myc antiserum. When CHO-K1 cells were incubated with increasing copper concentrations, the APP levels and p3 levels increased significantly (APP: cAPP to 295%, sAPP695 to 475% and sAPP751/770 to 275% with 10 μM copper and p3 to 154% with 10 μM copper; FIG. 4a-e; Table 1). At the same time, the Aβ production was drastically reduced below the detection limit (FIG. 4d,e). In the presence of copper chelating agents, the sAPP695 secretion was 170% (FIG. 54b,c), the sAPP751/770 secretion was 190% (FIG. 4b,c; Table 1) and the p3 secretion was 171% (FIG. 4d,e; Table 1).

Furthermore, the relative level of sAPP694, p3 and Aβ, which were immunoprecipitated from conditioned medium of CHO-K1 cells (a) and CHO-CUR3 cells (b), were quantified. The result is shown in FIG. 5. The increase in sAPP695 (black squares) reached a maximum at 10 μM Cu(II) (a) in K1 cells and at 50 μM Cu(II) (b) in CUR3 cells; thereafter there was a decrease. The change of the sAPP level (open triangles) occurred later as compared to the changes with the p3 protein which reached the maximum level with somewhat lower Cu(II) dosages, which is most likely due to a half life longer as compared to sAPP. In contrast thereto, the Aβ levels (blacks squares) increased drastically in both cell lines with Cu(II) concentrations below the basal level. The first data point was determined with the basal copper concentration (0.8 μM Cu(II)).

### EXAMPLE 6

### Identification of Copper Agonists for APP with Inhibitory Effect on the Release of the Amyloid Aβ Peptide

The copper-binding peptide of APP and APLP2 (homologous protein of APP which like APP and APLP1 belongs to the APP gene family), APPN262 (an artificially produced C-terminal shortened form of APP which consists of the N-terminal 262 residues of APP) and further artificially produced APP forms which are successively reduced by individual domains from the C terminus), the copper binding site-bearing variants of APP and APLP2 and fragments thereof were contacted with a differing concentration of Zn(II). The employed Zn²⁺ concentrations were 10 μM, 50 μM, 100 μM and 200 μM. Thereafter, it was studied from which concentration of Zn(II) the release of the Aβ peptide can be inhibited. The following experiments were carried out for this purpose:

(1) CHO cells which were stably transfected with human APP695 were incubated in MEM medium with the above-mentioned concentrations of Zn(II) (10 to 200 μM in PBS). CHO cells with copper ions (10 to 50 μM in PBS) or without any addition were incubated as controls. The production of Aβ (total), Aβ40 and Aβ42 was determined qualitatively and quantitatively with polyclonal and monoclonal antibodies. APP, Aβ and p3 were detected according to biosynthetic labeling with ³⁵S methionine as followed. The stably transfected CHO cells, which were incubated as mentioned above with the respective substances, were incubated for four hours with 220 μCi ³⁵S methionine as follows. Conditioned medium and cells were harvested and the dissolved proteins were used for the immunoprecipitation. The protein concentrations were determined beforehand using the protein detection kit from BioRad company and the amount of incorporated radioactivity was determined via scintillation counting. The cells were lyzed in extraction buffer (50 mM Tris-HCl, pH 7.5, 150 mM-NaCl, 2 mM EDTA, 2% triton X-100, 2% NP40, 1 mM PMSF, 10 μg/ml aprotinin and 10 μg leupeptin). Cell debris was centrifuged off at 13.000×g for 10 minutes and the supernatant was adjusted to 25 mM Tris-HCl, pH 8.5, 1 mM PMSF, 10 μg/ml aprotinin, 10 μg/ml leupeptin, 0.5% triton X-100 and 0.5% NP 40. The protein solution was diluted at a concentration of 1:2 with 100 mM Tris-HCl, pH 7,5, 300 mM NaCl and 4 mM EDTA. The supernatants were incubated with the above mentioned polyclonal APP and Aβ antibodies overnight at 4° C., the immunocomplexes formed were isolated with protein A sepharose and analyzed according to the method described in Ida et al., J. Biol. Chem. 271, pp. 22908-22914.

It showed that the Aβ production could be reduced drastically with a concentration from 50 μM Zn(II). A lowered secretion of APP in CXHO-K1 cells and in CUR3 cells showed with Zn(II) concentrations above 50 μM, in the case of an Aβ production reduced by 60% it showed with 50 μM Zn(II) for CUR3 cells (K1: 20%), by 90% it showed with 100 μM Zn(II) (K1:305) and by 99% it showed with 200 μM Zn(II) (K1: 60%). There was no further decrease of the Aβ production above 200 μM Ca(II), the detection limit having been reached here.

(2) Primary neurons were isolated from transgenic mice which expressed the Aβ peptide with the human sequence, and their Aβ production was determined as described in (1).

(3) The above mentioned Zn(II) concentrations in PBS were applied to transgenic mice orally, i.v., i.p., s.c. and i.c. (see (2)) and the Aβ production was determined in the CNS and in the blood (which was withdrawn from the caudal vein) as outlined above in (1). The serum obtained from the caudal vein of the mice (150-200 μl) is filled using PBS buffer up to a volume of 500 μl and incubated with 5 μg monoclonal antibody WO-2 (Ida et al., supra) and 20 μl of a 1:1 suspension of protein G sepharose overnight at 4° C. on an overhead shaker. The resulting supernatant is then incubated with polyclonal antibodies (22734/6) against APP and the immunocomplexes are analyzed as described above in (1). The Aβ immunocomplexes are separated by means of 12% bis-tricin Novex gels in accordance with the instructions of the manufacturer and the relevant molecular weight range is analyzed after the transfer of the proteins to a nitrocellulose filter (380 mA at 4° C. for 40 minutes) with the monoclonal antibody WO-2 using the ECL technique according to Ida et al. The result here was also that a decrease of the Aβ production could be obtained from a concentration of 50 μM Zn(II).

1. Use of the copper agonist which is characterized in that it binds to the copper binding site of the amyloid precursor protein (APP) and/or reduces or prevents the release of the amyloid Aβ peptide for the prevention or treatment of Alzheimer's disease.

2. Use according to claim 1, wherein the copper agonists are divalent metal ions, oligopeptides, oligonucleotides, oligosaccharides, nucleotide analogs, chemical substance libraries or low molecular natural substances from microorganisms or plants.

3. A method of identifying a copper agonist which binds to the copper binding site of APP and/or exerts an inhibitory effect on the release of the amyloid Aβ peptide, which is characterized by the steps of:
(a) contacting APP or a fragment thereof carrying the copper binding site with various concentrations of a compound which potentially has the above effect,
(b) detecting a decrease of the Aβ protein in mammals expressing the amyloid Aβ peptide.

4. The method according to claim 5, wherein the decrease of the Aβ peptide is detected by means of ELISA or immunoprecipitation from cell culture systems.

5. The method of identifying a copper agonist which binds to the copper binding site of APP and/or exerts an inhibitory effect on the release of the amyloid Aβ peptide which is characterized by the steps of:
(a) contacting of APP or a fragment thereof carrying the copper binding site with a dissolved or immobilized substance library or with low molecular substances from microorganisms and/or plants,
(b) when a dissolved substance library or liquid low molecular substances are used immunoprecipitation of the competitive or non-competitive copper binding site/ligand complex from the solution with antibodies specific to APP or the fragment thereof or, when an immobilized substance library is used, release of the ligand from the copper binding site/ligand complex by the addition of copper salts,
(c) identification of the ligand, and
(d) selection of ligands which after binding to the copper binding site of APP exert an inhibitory effect on the release of the amyloid Aβ peptide, wherein step (d) can optionally precede step (c).

6. The method according to claim 5, wherein step (d) comprises the incubation of mammalian cells stably transfected with APP695 with the ligand obtained from steps (a) to (c) and the determination of the production of the amyloid Aβ peptide with polyclonal or monoclonal antibodies.

7. The method according to claim 5, wherein step (d) comprises the administration of the ligand obtained from steps (a) to (c) to transgenic mammals which express the human amyloid Aβ peptide, the collection of a sample from the CNS or the blood and the determination of the production of the amyloid Aβ peptide with polyclonal or monoclonal antibodies.
